(19) United States
(12) Patent Application Publication
(10) Pub. No.: US 2022/0146199 A1
(43) Pub. Date: May 12, 2022

(54) Device and Method for Controlled Environment Cure

(71) Applicant: Chimera Technology LLC, Palatine, IL (US)

(72) Inventor: Geoffrey A. Bruder, Rocky River, OH (US)

(21) Appl. No.: 17/521,616

(22) Filed: Nov. 8, 2021

Related U.S. Application Data
(60) Provisional application No. 63/110,992, filed on Nov. 7, 2020.

(51) Int. Cl. F26B 21/10; F26B 5/04; A61L 2/10

(52) U.S. Cl. CPC F26B 21/10; A61L 2202/11; A61L 2/10; F26B 5/042

(57) ABSTRACT

A plant drying and curing device comprises a plurality of plant curing zones. A control system is configured to control one or more environmental conditions in each of the plurality of plant curing zones, and a receiver is configured to receive input data and process data according to a prescribed algorithm to control the one or more environmental variables.

### FIELD OF THE INVENTION

The present invention relates to a device and method for curing cannabis plants. More specifically, the present invention relates to a device and method for curing cannabis plants that utilizes a multi-zone cure environment which is modularly expandable, hermetically sealed and able to sustain vacuum.

### BACKGROUND OF THE INVENTION

As cannabis plants are processed for consumption, drying and curing methods are utilized to complete the drying of the flower and allow the terpenes and cannabinoids to evolve. During this process, which lasts around 4 weeks, the plant material is susceptible to degradation by light, oxygen, and biological contamination. The state-of-the-art technology for resolving these issues is either a low-cost sealed container, such as a bucket, or an expensive environmental control container with strict limitations. The low-cost option for the cure process requires manual agitation and renewal of internal environment, which is time consuming and costly. The available controlled environment options are not hermetic, are limited to flowing cover gases, and are tailored for niche product lines rather than mass production. A need therefore exists for a curing device and method that is modularly expandable, can be sealed and able to sustain a vacuum, and that can use standard industry hardware allowing it to be integrated into a building control system or alternatively to be used as a standalone controller.

### SUMMARY OF THE INVENTION

According to one aspect of the invention, a plant drying and curing device comprises a plurality of plant curing zones. A control system is configured to control one or more environmental conditions in each of the plurality of plant curing zones, and a receiver is configured to receive input data and process data according to a prescribed algorithm to control the one or more environmental variables. In an embodiment, each of the plurality of plant curing zones is hermetically sealed and isolated such that each zone can be individually controlled to desired environmental conditions. In an embodiment, each zone can be maintained at, below, or above atmospheric pressure. In an embodiment, each zone is sealed such that inert cover gases with small molecular sizes, such as helium, may be contained at or above atmospheric pressure.

In an embodiment, the plant drying and curing device can be integrated into a building control system. In another embodiment the plant drying and curing device can be a standalone device. In an embodiment, one or more of the zones includes an Ultraviolet (UV) light source mounted such that plant material disposed with the one or more zones can be biologically decontaminated during the drying and curing process. In an embodiment, decontaminating agents such as Ozone and Reactive Oxygen are introduced to decontaminate the plant matter and remove mold. In an embodiment, atomized scent or flavoring additives are added to one or more zones at or above atmospheric pressure. In a further embodiment, pre-dried air is constantly purged through the cure zone during the drying phase in order to rapidly remove plant material moisture.

According to another aspect of the invention, a modular plant drying and curing device comprises a plurality of modular plant curing zones. A control system disposed external to the plurality of modular plant curing zones is configured to control one or more environmental conditions in each of the plurality of modular plant curing zones, and a receiver disposed external to the plurality of modular plant curing zones is configured to receive input data and process data according to a prescribed algorithm to control the one or more environmental variables. In an embodiment, the modular plant drying and curing device includes a condenser, and an air circulation loop configured to carry air from one or more of the plurality of modular plant curing zones through the condenser so that terpenes and water are collected from the one or more of the plurality of modular plant curing zones and condensed in the condenser. In an embodiment the modular plant drying and curing device includes one or more liquid storage tanks for storing a liquid selected from the group consisting of: terpenes collected in the condenser, flavorings, essential oils, scents, or combinations thereof. In an embodiment, the liquid is introduced into the one or more of the plurality of modular plant curing zones.

### BRIEF DESCRIPTION OF THE DRAWINGS

FIG. 1 illustrates a schematic block diagram for an exemplary embodiment of a Controlled Environment Cure method.

FIG. 2 illustrates a configuration of an internal layout of one zone of an exemplary embodiment of a dry/cure device including circulating fans, heaters, UV lights, an ozone generator, and structural framing.

FIG. 3 illustrates an exemplary control system according to an embodiment.

FIG. 4 illustrates an exemplary embodiment of a single dry/cure zone including the exemplary control system.

FIG. 5 illustrates an exemplary embodiment of a single dry/cure zone including an exemplary condenser and air circulation loop.

### DETAILED DESCRIPTION

The curing process for cannabis flower requires either a low-cost sealed container, such as a bucket, or an expensive environmental control container with strict limitations. The proposed invention is a multi-zone cure environment which is modularly expandable, hermetically sealed and able to sustain vacuum, and utilizes standard industry hardware which can be integrated into a building control system or use a standalone controller.

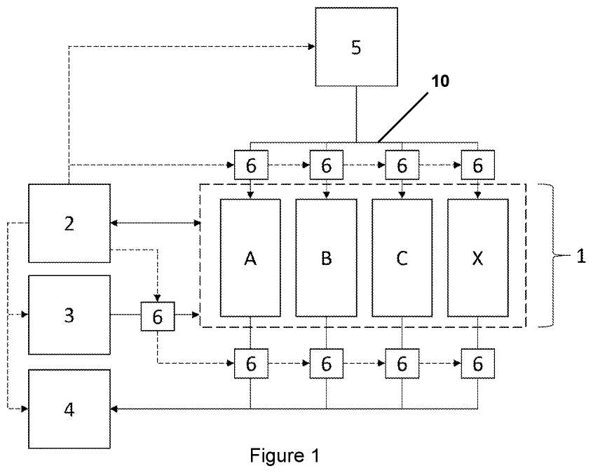

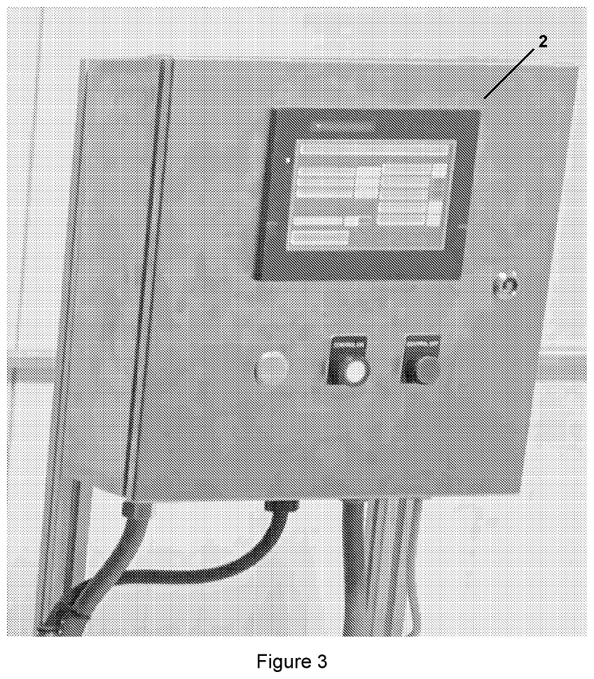

An exemplary embodiment of a plant curing device 10 is shown in FIG. 1, and includes a plurality of plant curing zones 1, indicated on the diagram as zone A, zone B, zone C, and zone X. Although four zones are shown the number of zones is not limited to four and can be any number of zones more or less than four, for example without limitation, two, three, four, five, six, seven, eight, nine, ten, eleven, twelve, thirteen, fourteen, fifteen, sixteen, seventeen, eighteen, nineteen, twenty, or more. In an embodiment the plurality of zones is maintained at desired environmental conditions by a single controller 2. An example controller 2 is shown in FIG. 3.

The controller 2 uses feedback (represented by the double arrow) from sensors (not shown) disposed within each of the plurality of plant curing zones 1 to control external devices 3, 4 that control one or more environmental conditions within the plurality of plant curing zones 1. For example without limitation, the external devices 3, 4 can provide a supply of gases or liquids, such as a supply of an inert gas or a supplemental terpene supply. The external devices 3, 4 can provide a vacuum supply, or a dry air supply, for example drawn from a tank of dry air 5.

In an embodiment the external devices 3, 4 can control the temperature of each of the plurality of plant curing zones 1. For example without limitation, in an embodiment each of the zones 1 is maintained at a temperature in a range between 50 and 70 degrees Fahrenheit. Keeping the temperatures in this range and generally below 70 degrees Fahrenheit limits the volatilization of terpenes and cannabinoids because, for example, terpenes begin to evaporate at about 70 degrees Fahrenheit. Temperatures higher than this range have also been found to damage plant material and are therefore undesirable.

In an embodiment the external devices 3, 4 can control the pressure within each of the plurality of plant curing zones 1. For example without limitation, in an embodiment a level of vacuum in one or more of the zones 1 is maintained at 25 Torr. It has been found that higher vacuum levels lower the water volatilization temperature, which allows plant material, for example cannabis flowers, to dry more quickly while also limiting damage to the plant material.

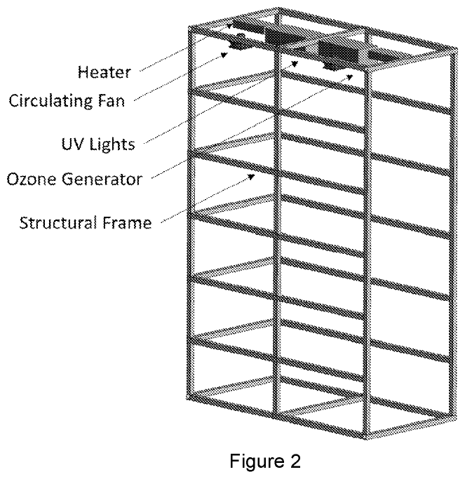

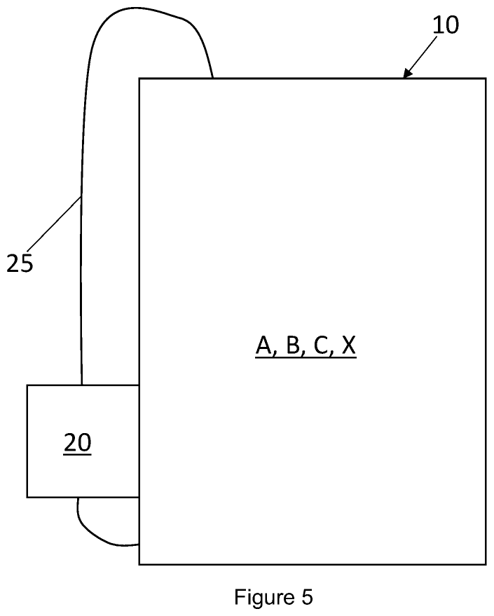

Further external or internal devices, for example without limitation, such as ultraviolet (UV) lights, Ozone generators, heaters, a circulating fan, or chillers may also be controlled and are shown in FIG. 2 in exemplary relation to a structural frame. In an embodiment, contamination of plant material in each of the plurality of plant curing zones 1 is mitigated by the introduction of Ozone or Reactive Oxygen into the zones. In another embodiment, contamination is mitigated by the introduction of chlorine dioxide. In a further embodiment, UV lights are included in one or more of the plurality of plant curing zones 1 to clean the air in each zone. The UV lights can also be used within the recirculation loop 25 (see FIG. 5) to clean the air within the recirculation loop 25.

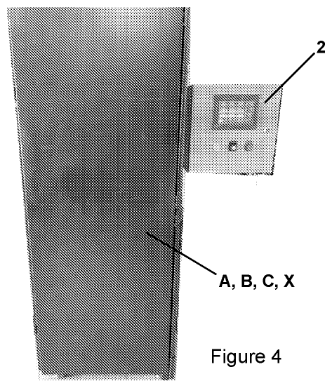

Each external device 3, 4 is connected to a hermetically sealed zone A, B, C, X of the plurality of plant curing zones 1 through a solenoid 6 that is controlled to open or close as needed at the appropriate timing in a cycle sequence in order to maintain the appropriate environment within the zone A, B, C, X. An exemplary zone A, B, C, X and controller 2 are shown in FIG. 4.

Referring to FIG. 5, in an embodiment the device 10 includes a condenser 20 mounted on an air circulation loop 25. The condenser 20 in an embodiment is a multi-stage condenser having a first stage that, for example, operates at about 32 degrees Fahrenheit and a second stage that, for example, operates at about −112 degrees Fahrenheit. In the stage condenser 20, water with a majority of the terpenes would be captured in the first stage. The water and terpenes can be separated so that the terpenes can be reintroduced back into the plant material. The second stage of the condenser 20 would collect the remainder of the volatilized terpenes. The condenser 20 can have other operating temperatures in other embodiments.

The tank 5 (or another like it) in FIG. 1 can be used as a liquid storage tank. For example, in an embodiment, the tank 5 is a tank for storing terpenes or other additives, wherein the terpenes or other additives are, for example, atomized and introduced into one or more of the plurality of zones 1. For example, in an embodiment, each of the zones A, B, C, X is pressurized and during the pressurizing step the terpenes and/or other additives are introduced. The terpenes and/or other additives can be atomized within each of the zones or atomized external to the zones. Atomization can be achieved with a nozzle or sprayer as is known in the art.

The addition of terpenes and/or other additives is useful because it has been found that cannabis flower can be enhanced by the addition of terpenes. The terpenes introduced in this process could come from the terpenes collected in one or both stages of the condenser 20, and so would actually be reintroduced into the plant material. Other additives include flavorings or essential oils, for example without limitation, vanilla, blueberry, lavender, and other flavors and scents. In an embodiment the liquids in the tank 5 are introduced into one or more of the plurality of plant curing zones 1 at a pressure of, for example without limitation, 15 pounds per square inch (psi) over atmospheric pressure. Introduction of the liquids at an elevated pressure helps to force volatilized components into the plant material, for example, into the cannabis flower.

The device 10 as described herein provides many benefits over existing devices. For example, the device 10 includes features as described above that reduce the dry/cure time from about 1 week from about 5 weeks for existing devices and methods. The device 10 can do this while maintaining 100% of the natural plant terpenes, whereas other methods result in a loss of terpenes, for example, hang drying loses over 20% of the terpenes. Flower contamination is limited by containing the plant material within each zone A, B, C, X, which limits handling of the plant material. Limiting the handling in this way increases the potency of the plant material because trichromes are better maintained on the exterior of the cannabis flower. Existing systems have all the parts are integrated into a single vessel, which requires the purchase of multiples of expensive components such as air compressors, vacuum pumps, and control units when expanding. In contrast, the device 10 is modular and expandable with minimal additional investment because the major components of the device 10 are disposed outside of the dry/cure zones A, B, C, X so expansion requires only the addition of more zones without the expensive components. Piping and connections for carrying gases and liquids between the components can be modularly expanded as is known in the art, for example through the use of well-known connectors, pipe fittings, and manifolds.

### INDUSTRIAL APPLICABILITY

A plant drying and curing device comprises a plurality of plant curing zones that can be modular. A control system is configured to control one or more environmental conditions in each of the plurality of plant curing zones, and a receiver is configured to receive input data and process data according to a prescribed algorithm to control the one or more environmental variables. The plant drying and curing device can be manufactured in industry for use by consumers.

Numerous modifications to the present invention will be apparent to those skilled in the art in view of the foregoing description. It is not desired to limit the invention to the exact construction and operation shown and described, and accordingly, all suitable modifications and equivalents may be resorted to, falling within the scope of the invention. Accordingly, this description is to be construed as illustrative only of the principles of the invention and is presented for the purpose of enabling those skilled in the art to make and use the invention and to teach the best mode of carrying out same. The exclusive rights to all modifications which come within the scope of the appended claims are reserved. All patents, patent publications and applications, and other references cited herein are incorporated by reference herein in their entirety.

I claim:

1. A plant curing device, comprising:
a plurality of plant curing zones;
a control system configured to control one or more environmental conditions in each of the plurality of plant curing zones; and
a receiver configured to receive input data and process data according to a prescribed algorithm to control the one or more environmental variables.

2. The device of claim 1, wherein the plurality of plant curing zones are hermetically sealed and isolated from one another such that each of the plurality of plant curing zones can be individually controlled to desired environmental conditions.

3. The device of claim 1, wherein one or more of the plurality of plant curing zones are maintained at below atmospheric pressure.

4. The device of claim 1, wherein one or more of the plurality of plant curing zones are maintained at or above atmospheric pressure.

5. The device of claim 4, further comprising an atomized scent introduced into one or more of the plurality of plant curing zones.

6. The device of claim 4, further comprising a flavoring introduced into one or more of the plurality of plant curing zones.

7. The device of claim 1, wherein each of the plurality of curing zones is sealed such that inert cover gases with small molecular sizes may be contained at above atmospheric pressure.

8. The device of claim 1, wherein the control system is configured to be integrated into a building control system.

9. The device of claim 1, wherein the control system is configured to be a standalone system.

10. The device of claim 1, further comprising an Ultraviolet (UV) light source configured to biologically decontaminate plant material disposed within the plurality of plant curing zones.

11. The device of claim 1, further comprising a plant for curing, wherein the plant is cannabis.

12. The device of claim 1, further comprising a decontaminating agent introduced into the plurality of plant curing zones to decontaminate plant matter and remove mold.

13. The device of claim 11, wherein the decontaminating agent is selected from the group consisting of Ozone, Reactive Oxygen, chlorine dioxide, or a combination thereof.

14. The device of claim 1, further comprising:
a drying phase of operation; and
pre-dried air that is constantly purged through the plurality of plant curing zones during the drying phase in order to rapidly remove plant material moisture.

15. A modular plant curing device, comprising:
a plurality of modular plant curing zones;
a control system disposed external to the plurality of modular plant curing zones, the control system configured to control one or more environmental conditions in each of the plurality of modular plant curing zones; and
a receiver disposed external to the plurality of modular plant curing zones, the receiver configured to receive input data and process data according to a prescribed algorithm to control the one or more environmental variables.

16. The device of claim 15, wherein the control system is configured to be integrated into a building control system.

17. The device of claim 15, further comprising:
a condenser; and
an air circulation loop configured to carry air from one or more of the plurality of modular plant curing zones through the condenser so that terpenes and water are collected from the one or more of the plurality of modular plant curing zones and condensed in the condenser.

18. The device of claim 17, further comprising one or more liquid storage tanks for storing a liquid selected from the group consisting of: terpenes collected in the condenser, flavorings, essential oils, scents, or combinations thereof.

19. The device of claim 18, wherein the one or more of the plurality of modular plant curing zones are maintained at or above atmospheric pressure.

20. The device of claim 19, further comprising the liquid introduced into the one or more of the plurality of modular plant curing zones.
